(19) United States
(12) Patent Application Publication
(10) Pub. No.: US 2017/0135855 A1
(43) Pub. Date: May 18, 2017

(54) PATIENT WARMING SYSTEM

(71) Applicant: VITAHEAT MEDICAL, LLC, Rolling Meadows, IL (US)

(72) Inventors: Ann C. Stefan, Deer Park, IL (US); Stephen B. Smith, Antioch, IL (US); Victor J. Orler, Hinsdale, IL (US); James T. Gaffney, Westmont, IL (US); William T. Renwick, Hinsdale, IL (US); Robin A. Wedell, Naperville, IL (US); David K. Mezera, Issaquah, WA (US); Stephen D. Dunlap, Bellevue, MI (US); James A. Medsker, Lawton, MI (US)

(73) Assignee: VITAHEAT MEDICAL, LLC, Rolling Meadows, IL (US)

(21) Appl. No.: 14/649,815

(22) PCT Filed: May 21, 2015
(86) PCT No.: PCT/US15/31923

§ 371 Date: Jun. 4, 2015

(51) Int. Cl. A61F 7/08; A61F 7/00

(52) U.S. Cl. CPC A61F 7/08; A61F 7/007; A61F 2007/0078; A61F 2007/0095

(57) ABSTRACT

This disclosure relates to warming systems and methods of using such warming systems. The warming systems include a heating pad and a controller. The heating pad includes a pouch having a substrate contained therein and a heating element disposed on the substrate. The heating element may include conductive ink, it may be a polymeric sheet doped with one or more metals, or it may be a fabric impregnated with one or more metals. The heating pad may also include thermocouples, pressure sensors, and temperature change sensors.

### BACKGROUND

1. Field of the Invention

The present disclosure generally relates to warming systems.

More particularly, the disclosure relates to heating pads that may be used in clinical settings.

2. Description of the Related Art

Current patient warming systems primarily utilize forced heated air, which is circulated through a disposable patient covering on top of the patient. These over-body systems provide limited application for many surgical procedures and release heated air into the operating room. They are also noisy and thus interfere with the communication of the surgical team. Additionally, such systems have a limitation in that they require connection to an AC power outlet and thus they are not portable.

### BRIEF SUMMARY

The present disclosure relates to warming systems and methods of using the same. In one embodiment, a warming system is disclosed comprising a heating pad and a controller, the heating pad comprising a substrate comprising a heating element disposed under a covering. The heating element comprises conductive ink and one or more thermocouples, wherein the one or more thermocouples have diameters less than about 0.13 mm. The warming system also comprises an electric cord in communication with the heating element and the controller, the controller comprising a battery and being configured to increase or decrease an amount of an electric current being supplied to the heating element. The substrate additionally comprises one or more discrete lines of the conductive ink forming a pattern and each line of conductive ink has a thickness of less than about 12 μm.

In another embodiment, the disclosure relates to a method of warming a body part of a patient. The method comprises the steps of providing a warming system, wherein the warming system comprises a heating pad and a controller, the heating pad comprising a substrate comprising a heating element disposed under a covering, wherein the heating element comprises one or more thermocouples. The warming system further comprises an electric cord in communication with the heating element and the controller, the controller comprising a battery and being configured to increase or decrease an amount of an electric current being supplied to the heating element. The substrate comprises one or more pressure sensors connected via a conduit to one or more transducers, the transducers being in electronic communication with the controller. The method also includes the step of contacting a portion of the heating pad with the body part of the patient, the body part of the patient applying pressure to one or more of the pressure sensors causing a pressure change wherein air from within the one or more pressure sensors flows through the conduit and into the one or more transducers. Additionally, the method comprises the steps of sending a communication from the one or more transducers to the controller to alert the controller of the pressure change and then supplying electric current only to a portion of the heating element where the pressure change was detected and not to any other portion of the heating element.

The disclosure further provides a warming system comprising a heating pad and a controller, wherein the heating pad comprises a substrate comprising a heating element disposed under a covering and the heating element comprises one or more thermocouples, wherein the one or more thermocouples have diameters less than about 0.13 mm. The system also comprises an electric cord in communication with the heating element and the controller, the controller comprising a battery and being configured to increase or decrease an amount of an electric current being supplied to the heating element. The substrate additionally comprises one or more pressure sensors connected via a conduit to one or more transducers, the transducers being in electronic communication with the controller.

Additional features and advantages of the disclosure will be described hereinafter that form the subject of the claims of this application. It should be appreciated by those skilled in the art that the concepts and the specific embodiments disclosed may be readily utilized as a basis for modifying or designing other embodiments for carrying out the same or similar purposes of the present invention. It should also be realized by those skilled in the art that such equivalent embodiments do not depart from the spirit and scope of the invention as set forth in the appended claims.

### BRIEF DESCRIPTION OF THE DRAWINGS

A detailed description of embodiments of the invention is hereafter described with specific reference being made to the drawings in which:

FIG. 1 shows an embodiment of a warming system according to the present disclosure;

FIG. 2 shows various layers associated with an embodiment of a heating pad;

FIG. 3 shows an embodiment of a heating element;

FIGS. 4A-4C show alternate embodiments of a heating pad in comprising slits;

FIG. 5 shows various layers of an embodiment of a heating pad in accordance with the present disclosure;

FIG. 6 shows an embodiment of a heating pad comprising openings;

FIG. 7 shows a heating pad comprising multiple pressure sensors and temperature change sensors;

FIG. 8A shows a pressure sensor in accordance with an embodiment of the present disclosure;

FIG. 8B shows an alternative embodiment of a pressure sensor in accordance with an embodiment of the present disclosure;

FIG. 9 shows various layers associated with an embodiment of a heating pad; and

FIG. 10 shows an embodiment of a warming system according to the present disclosure.

### DETAILED DESCRIPTION

Various embodiments are described below with reference to the drawings in which like elements generally are referred to by like numerals. The relationship and functioning of the various elements of the embodiments may be better understood by reference to the following detailed description.

However, embodiments of the invention are not limited to those illustrated in the drawings. It should be understood that the drawings are not necessarily to scale, and in certain instances details may have been omitted that are not necessary for an understanding of embodiments disclosed herein, such as conventional fabrication and assembly.

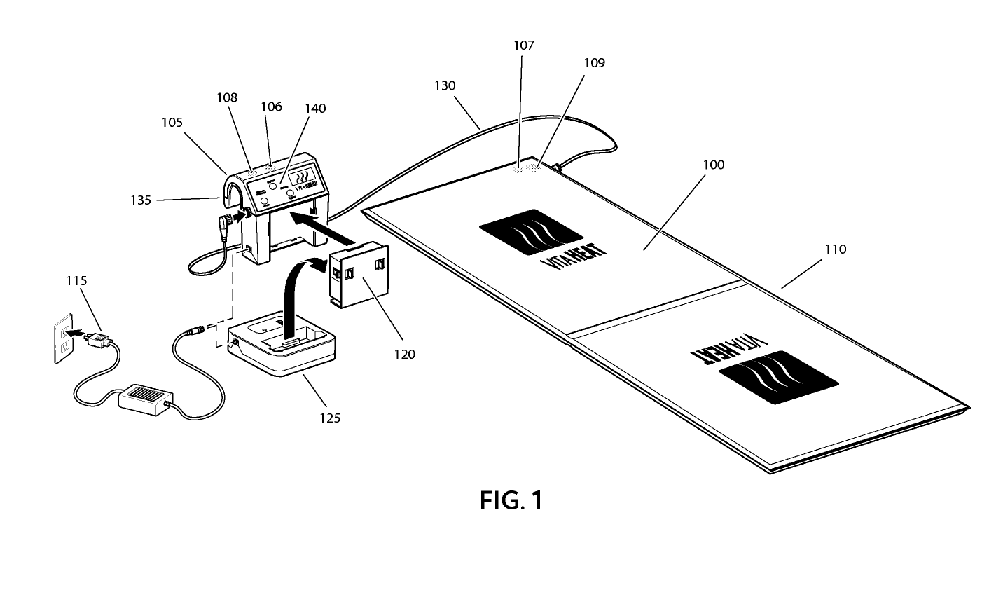

The present application generally relates to warming systems and various different aspects thereof. In some embodiments, such as shown in FIG. 1, the warming system comprises a heating pad, which includes a cover (100) or pouch having a front and back cover, a substrate, such as an insulating pad, disposed within the pouch, and a heating element disposed on the substrate. The warming system also comprises a power source and a controller (105) for operating the heating pad. In some embodiments, the pouch is excluded and the warming system comprises a covering (100) for the substrate. The covering may cover the front and back sides of the substrate. The pouch and covering may be made from any suitable materials to provide a flexible barrier between the heating element and a patient in contact with the heating pad. In some embodiments, the pouch or covering comprises cloth or fabric materials.

In some embodiments, the heating pad is radio translucent, meaning that it does not appear on an X-ray image which may be taken of a patient in contact with the heating pad. While the heating pad may appear in an X-ray image in certain embodiments if the image was solely taken of the heating pad, when a patient is in contact with the heating pad and an X-ray image is taken of the patient, the heating pad does not appear in the X-ray image. In some embodiments, the heating system is portable.

As shown in FIG. 1, a power source may be coupled to the heating element to provide an electric current thereto and a power source may also be coupled to the controller (105). In certain embodiments, the warming system is a closed-loop control warming system comprising a controller (105) and a heating pad (110), which may be placed within a disposable blanket, pouch, or underneath a covering (100). The warming system may be a lightweight, portable system, meaning that it does not need to be plugged into an electrical outlet to receive power, although, for the purposes of illustration only, a plug (115) is shown in FIG. 1. Instead, the system may be powered by a battery (120), which can be a rechargeable battery. The controller may comprise a removable, rechargeable battery (120) or, in other embodiments, the battery may not be removable from the controller. In certain embodiments, a specialized key may be required to remove the battery from the controller. The controller (105) may comprise an integral battery charger for charging the battery during operation of the system or the battery may be removed and charged in a separate battery charging unit (125). In certain embodiments, the heating pad may be foldable to further improve the portability of the system. In some embodiments, the heating pad is disposable.

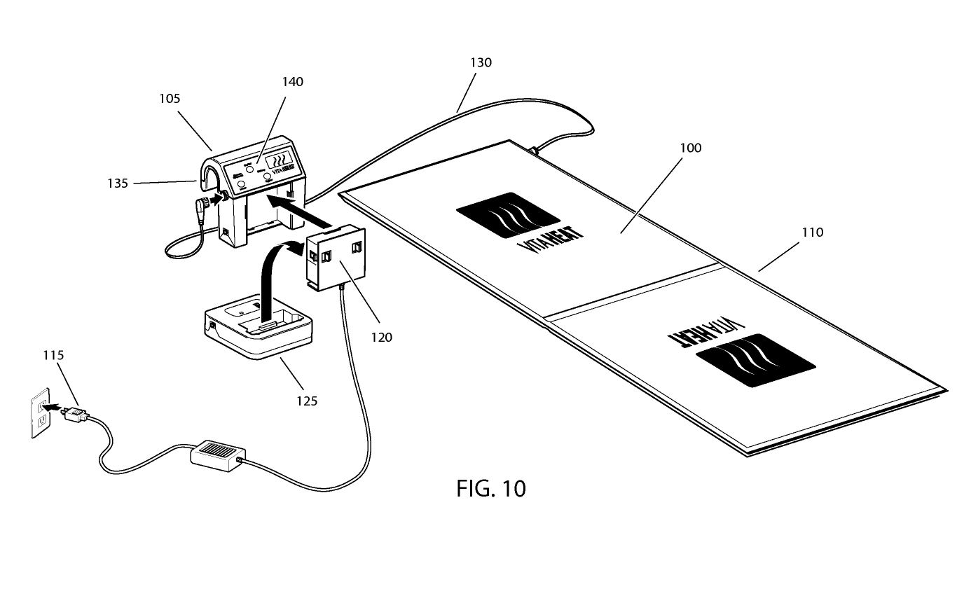

Instead of having the power source (115) directly connected to the controller (105) as in FIG. 1, the power source (115) may be connected directly to the battery (120) as can be seen in FIG. 10. In this embodiment, the AC power is provided via a modified battery housing (where the batteries have been removed from the housing). Although this embodiment still may include an AC/DC power brick, instead of it plugging in to the controller with a special plug, a battery housing may be used as a “plug”.

In some embodiments, the warming system may be used to warm a patient in a hospital and/or surgical center including, but not limited to, operating, recovery and emergency rooms, as well as on medical/surgical floors. The presently disclosed warming system may be used in products that extend beyond the clinical setting as well. Further, it is contemplated that the warming system may be used with patients being transported in an EMS (Emergency Medical System) ground or air vehicle.

The warming system (or any component thereof) may be powered by any known power source. In some embodiments, the warming system may be powered by an alternating current (AC) and/or a direct current (DC). In some embodiments, the power source is a battery, such as a lithium ion battery, and in other embodiments, the warming system may be plugged into an AC electrical outlet to receive power. In certain embodiments, the warming system may comprise both a battery and a power cord that may be plugged into an electrical outlet. In such an embodiment, a battery may power the heating element to warm a patient and at the same time, the power supplied from the electrical outlet may charge the battery. In some embodiments, a controller associated with the warming system may comprise an integral AC battery charger. In certain embodiments, the warming system, or a component thereof, such as the controller, may comprise a low battery power alert system, whereby a signal may be sent, such as a digital signal or an audible signal, to alert an operator that the battery of the system is low on power.

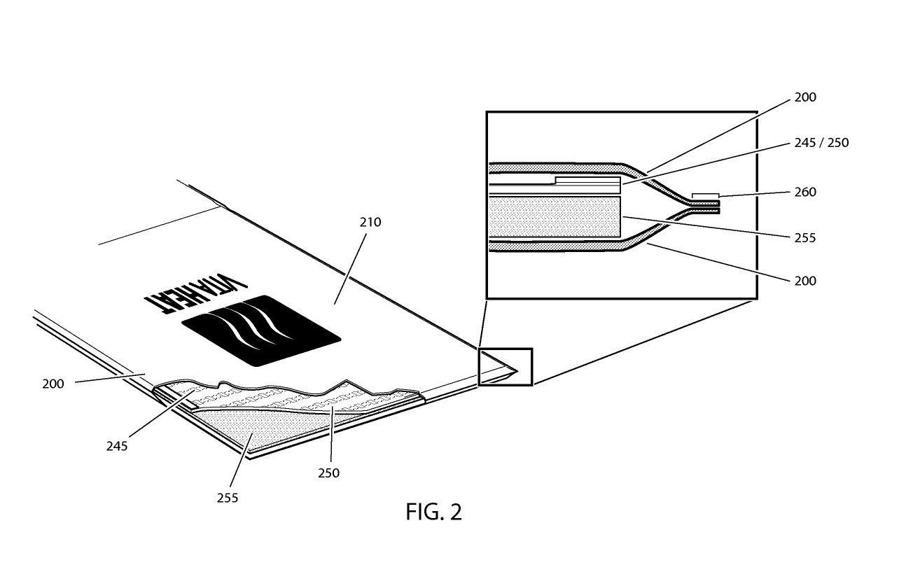

In some embodiments, such as shown in FIG. 2, the heating element comprises conductive ink (245). In such embodiments, the heating pad (210) comprises a substrate (250) and a flexible insulating pad (255), and the conductive ink (245) may be printed onto the substrate (250) and fixed (immobilized) on the substrate (250). The insulating pad (255) may, in some embodiments, comprise foam and/or gel. The substrate (250) may comprise any suitable material, for example, acetate, mylar, metal, paper, cloth, and any combination thereof, and the substrate may have any suitable construction and configuration, such as a square, rectangular, or circular configuration. The substrate may have sufficient flexibility such that it conforms to a patient's body or a patient's body part in contact therewith.

Also, a covering (200), pouch, blanket, or the like may be placed over the heating element to provide a barrier between the heating element and a patient. In some embodiments, an additional layer may be provided between the heating element and the cover or pouch. For example, the heating element may be disposed on a mylar substrate and an additional mylar layer, foam layer, gel layer, or layer comprising foam sandwiched between nylon, may be disposed on top of the heating element. In some embodiments, the layer between the cover and the heating element comprises about 0.04 inches of foam laminated between a looped nylon covering. In certain embodiments, the layer between the heating element and the cover may be referred to as a heat dissipating layer. Finally, as will be further described below, the heating pad may comprise a radio-frequency welded flange (260).

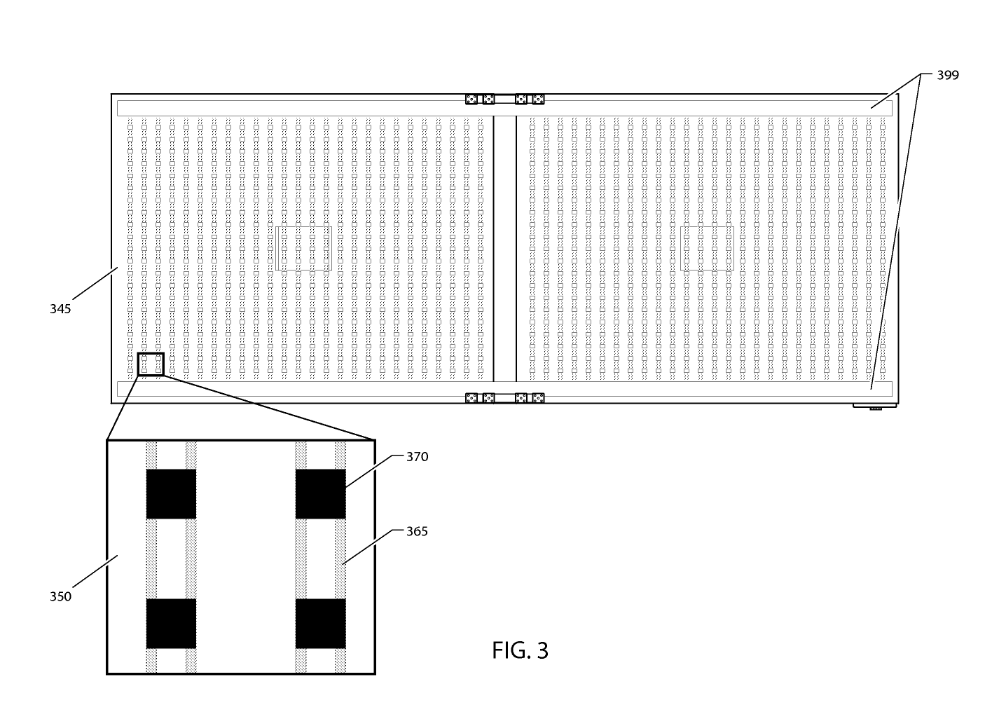

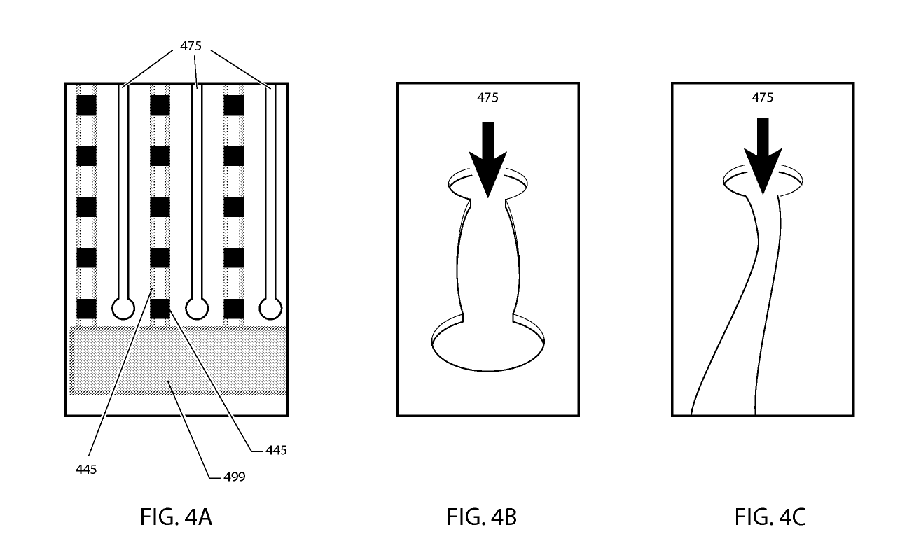

In some embodiments, the substrate may be rigid or somewhat inflexible such that conformance with a patient or patient's body part in contact therewith is not readily accomplished. However, as is shown in FIGS. 4A-4C, flexibility can be imparted to the substrate by cutting grooves, slits, or the like into the substrate. The slits or grooves (475) may be cut vertically, horizontally, and/or diagonally into the substrate between the printed ink (445). The slits do not need to be cut in straight lines and can comprise any shape, such as cross, circular, etc. FIG. 4A also depicts a power distribution conductor (499) which is described below in connection with FIG. 3.

The conductive ink printed onto the substrate is not limited and any known inks may be used. In some embodiments, the ink printed onto the substrate may be selected from carbon, silver, graphite, copper, and the like, and any combination thereof. The conductive ink may be fixedly disposed on the substrate using any suitable methods, such as a conventional printing press, a screen printing press, or an ink-jet printer. After the ink is printed onto the substrate, it may be cured and set using commonly known methods in the art, such as application of heat or UV light.

The conductive ink may be printed onto the substrate in any desired pattern. For example, the ink may be printed onto the substrate in a series of lines, circular patterns, rectangular patterns, square patterns, or any combination thereof. In some embodiments, such as shown in FIG. 3, the conductive ink (345) may be printed in the form of a grid on the substrate (350). For example, a silver ink (365) may be used to convey electricity and a carbon ink (370) may be used to convert the electricity to heat. Lines of silver ink (365) may be printed vertically along the substrate (350) and squares or lines of carbon ink (370) may be printed onto the substrate such that they intersect one or more lines of silver ink (365). Power distribution conductors (399) may be applied to the screen printed circuit to provide even distribution of power to the entire screen printed circuit. The conductors (399) may be applied to the screen printed traces by any fastening means including rivets, clamps, crimps, adhesive overlay, etc.

The size of the printed squares (or circles, rectangles, lines, or whatever pattern is used) may be selected by one of ordinary skill in the art.

In some embodiments, the size of the squares (or other selected pattern) is chosen to achieve even heat distribution. In one embodiment, the squares of carbon ink may have a length and width of about 0.25 inches and a thickness between about 8 μm and about 12 μm. In certain embodiments, the substrate may comprise squares having a larger area near the extremities and squares having smaller areas near the mid-section of the substrate. By printing the outer squares (or other shapes) with larger areas, this compensates for any increased heat loss along the edges of the heating pad.

The heating pad may also comprise features that allow for a zonal-type heating arrangement and/or detection of a patient or person coming into contact with the heating pad. For example, if the substrate of the heating pad was square-shaped and the square was divided into four quadrants, each quadrant may comprise one or more pressure sensors and/or temperature change sensors, which, in some embodiments, can be the thermocouples described below. If a body part of a patient contacted a particular quadrant of the heating pad, or any portion of the heating pad comprising a pressure (or temperature change) sensor, the sensor would detect the pressure (or temperature) change and communicate this change to the controller, for example. The controller may then cause an electric current to be supplied to the heating element near the section of the heating pad experiencing the pressure (or temperature) change, thereby raising the temperature of this section of the heating pad and warming the patient. In turn, any portion of the heating pad not experiencing the pressure (or temperature) change may not have an electric current supplied thereto, thereby conserving energy.

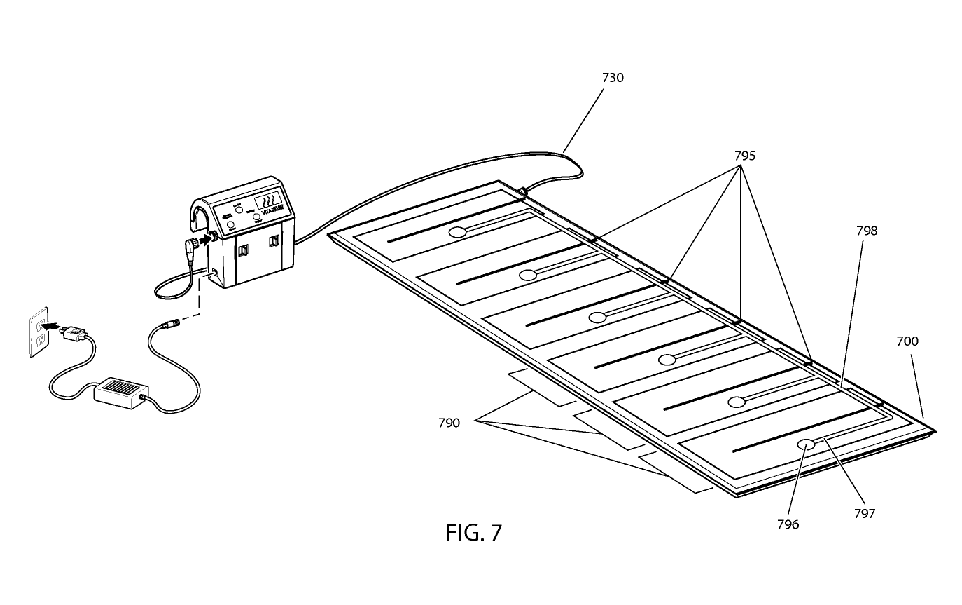

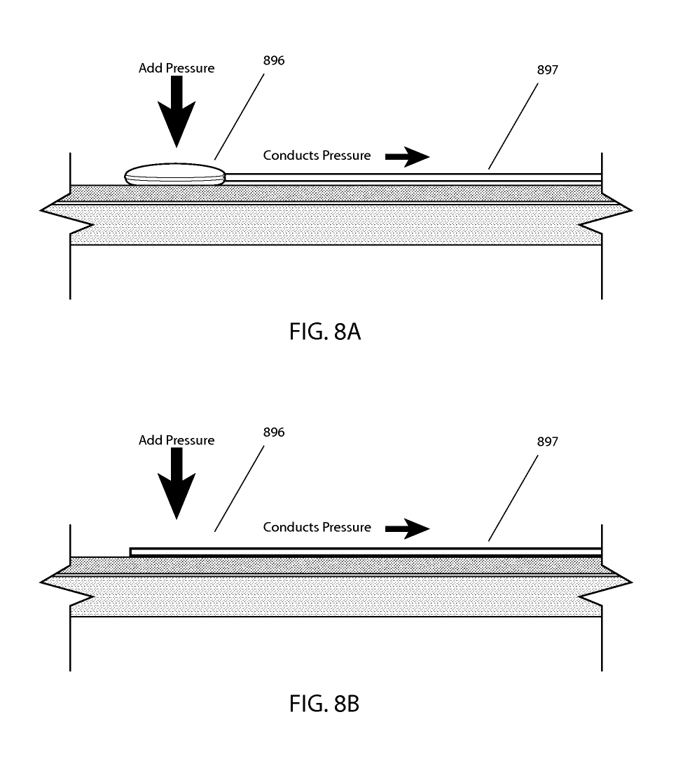

Any known pressure sensor may be used in connection with the warming system. In some embodiments, as depicted in FIG. 8, the pressure sensor (896) may be a radio translucent air pouch or air capsule. For example, a flexible sheet of polymeric material may be folded over, sealed along its edges, and a pocket of air may be formed therein. In some embodiments, the pressure sensor may be a flexible membrane that, when compressed, provides air pressure that the conduit delivers to the transducer. One or more of these air pouches/capsules (896) may be disposed at any location on the substrate. As seen in FIG. 7, each zone of the heating pad may comprise a pressure sensor (796) or each zone may comprise more than one pressure sensor. A hollow conduit (897) may be inserted into the air pouch (896) and an airtight seal may be formed at the point of entry. The other end of the conduit may be attached to a second conduit (see 798 in FIG. 7), which may be in communication with a transducer located, for example, at an edge of the heating pad, either within the pouch or on the outside of the pouch. When pressure is applied to the air pouch, some of the air within the pouch is forced out of the pouch and travels through the conduit (797), through the second conduit (798) to the transducer. When more than one pressure sensor is present (as in FIG. 7), each pressure sensor may comprise a conduit in communication with a transducer. When the transducer detects the airflow, it sends an electronic signal to the controller. In some embodiments, the signal is sent through a wire connecting the transducer and controller and in other embodiments, the signal is sent wirelessly. When the controller receives the signal, it causes an electric current to be supplied to the section of the heating element near the depressed air pouch. Since the pressure sensor and the conduit may comprise plastic or polymeric transparent materials, they can be radio translucent and thus excluded from X-ray images.

Just as one or more portions or sections of the heating pad may comprise a pressure sensor, one or more portions or sections (790) of the heating pad (700) may comprise a temperature change sensor, which, in some embodiments, can be the thermocouple (795) described below. When a part of a body contacts a portion of the heating pad, there will be a temperature change at that portion of the heating pad. A temperature change sensor (795) can sense this temperature change and communicate the same to a controller (705), for example, either wirelessly or through a wired connection (730) between the temperature change sensor and the controller. The controller may then cause an electric current to be supplied to the heating element near the section of the heating pad experiencing the temperature change, thereby raising the temperature of this section of the heating pad and warming the patient. In turn, any portion of the heating pad not experiencing the temperature change caused by patient contact may not have an electric current supplied thereto, thereby conserving energy.

It can thus be seen that by incorporating one or more pressure sensors and/or one or more temperature change sensors into the heating pad, the heating pad can have a zonal heating configuration and it can detect the presence of a patient or body part of a patient. In some embodiments, since an electric current is only sent to the portion of the heating pad contacting the patient, energy may be conserved since electric current is not supplied to portions of the heating pad not contacting the patient. Each zone of the heating pad may comprise one or more pressure sensors and/or temperature change sensors and each sensor can be independently in communication with the controller such that if one or more sensor were to fail or break down, the remaining sensors would continue to operate, thereby allowing the patient to continue to be heated. The warming system may comprise redundant temperature sensors and/or pressure sensors, which eliminates the possibility of automatic system shutdown due to the failure of a single sensor.

As shown in FIG. 8B, the pressure sensor (896) may comprise an elongated air tube that, in some embodiments, may stretch from one end of the heating pad to the opposite end. The elongated air tube may be flexible and/or crushable, similar to the air pouch, so that it can deform when pressure is applied thereto by a patient. While the air pouch shown in FIG. 8A can provide discrete, pin-point pressure sensing capabilities, an elongated air tube operates under the same principles as those disclosed in connection with the air pouch but it can sense pressure along the entire pad (wherever it is disposed) as opposed to a single exact location like the air pouch. The elongated air tube may be placed on the heating in any pattern and may cover one or more zones of the heating pad. The elongated air tube may communicate directly with one or more transducers in a fashion similar to that shown with the conduit (897) in FIG. 8A.

In some embodiments, the heating pad, or any component thereof, is radio translucent. In such embodiments, if the heating pad is placed near a body part of a patient and an X-ray is taken of that body part, no components of the heating pad (such as heating element, pressure sensor, temperature change sensor, etc.) will be visible in the X-ray image. If the heating element comprises printed ink, for example, carbon and silver ink, the X-rays may actually travel around the ink such that the ink is not visible in an X-ray image. Each line of printed ink of the heating element may comprise small ink particles adjacent one another and the X-rays may travel around the particles. Moreover, the printed ink may comprise extremely thin thicknesses to allow for radio translucency. For example, the thickness of a printed line of ink may be from about 1 μm to about 12 μm. In some embodiments, the thickness may be from about 5 μm to about 10 μm or from about 8 μm to about 12 μm. If a line of printed ink was thicker than 12 μm, for example, the line may not be radio translucent and may therefore interfere with an X-ray image.

The heating pad, substrate, and/or heating element may comprise a thermocouple for sensing temperature. In some embodiments, the thermocouple may have an extremely small diameter, such as from about 0.02 mm to about 0.13 mm. In one embodiment, the diameter is less than about 0.05 mm or less than about 0.03 mm. Such a small diameter will allow the thermocouple to be radio translucent. In some embodiments, the thermocouple may be weaved through the substrate. In some embodiments, the thermocouple may be disposed on the heating element. In any embodiment, the heating pad may comprise one or more thermocouples and each thermocouple may independently be in communication with the controller, either via a wired connection or a wireless connection. As noted above, the thermocouples can be temperature change sensors used for a zonal heating method where electronic current is only supplied to a portion of the heating element near one or more thermocouples sensing a temperature change, such as an increase in temperature. In some embodiments, the temperature change sensor (e.g. the thermocouple) is an analog sensor, which helps reduce sensitivity to interference with other equipment that may be in the operating room, for example.

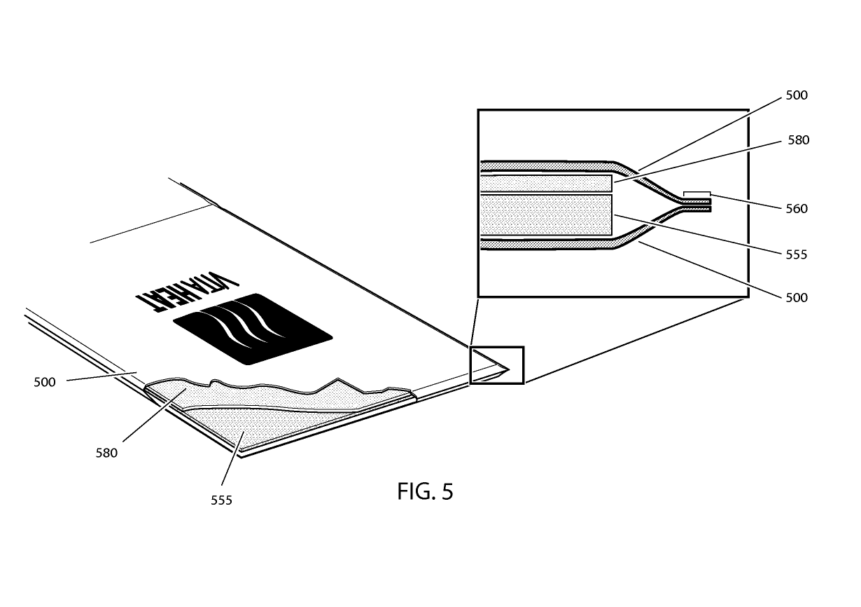

Instead of comprising conductive ink, in some embodiments, such as shown in FIG. 5, the heating element may comprise a flexible polymer sheet (580) doped with metals, such as carbon and silver. The sheet or film may comprise, for example, silicone and/or polyethylene. The sheet may also comprise one or more pressure sensors, thermocouples, and/or one or more temperature change sensors. The sheet (580) may be disposed between a cover (500) and an insulating pad (555). Finally, the heating pad may comprise a radio-frequency welded flange (560) on its cover to be used in connection with a manufacturing process where high energy RF waves are used to melt the cover material and join the top and bottom cover pieces.

In other embodiments, the flexible polymer sheet of FIG. 5 may be removed and replaced with an impregnated flexible fabric. In some embodiments, the fabric may be impregnated with steel, silver, nickel, and/or carbon, for example. In certain embodiments, the flexible fabric is a carbon fiber conductive fabric. In other embodiments, the fabric comprises nylon or polyester. One of ordinary skill in the art will appreciate various methods which can be used to impregnate the fabric with silver, carbon, etc., such as vacuum deposition. The impregnated fabric may act as the heating element and it may be placed within the pouch. The flexible fabric may additionally comprise one or more pressure sensors, thermocouples, and/or one or more temperature change sensors.

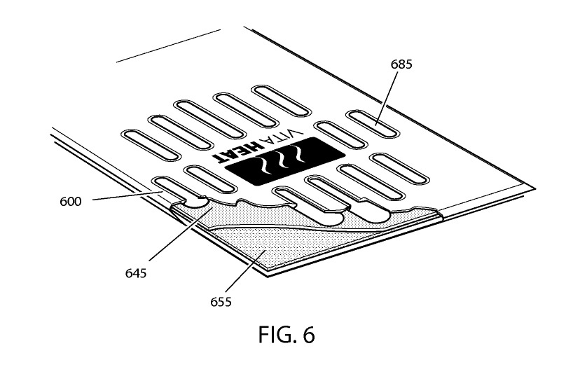

Regardless of the heating element being used, openings (685) or windows may be cut through the entire heating pad, to allow for greater flexibility and air flow, as shown in FIG. 6. If the heating element (645) comprises a flexible polymer sheet or impregnated flexible fabric, openings (685) may be cut directly through the cover (600), heating element (645), and insulating pad (655). If the heating element (645) comprises conductive ink, care should be taken to cut the openings (685) at locations on the heating pad that will not intersect or separate the conductive ink. For example, if lines of conductive ink are printed vertically on a substrate and spaced apart from each other, the openings (685) may be cut in between the vertical lines of ink. The openings (685) can have any shape, such as oval, circular, elliptical, rectangular, square, and the like.

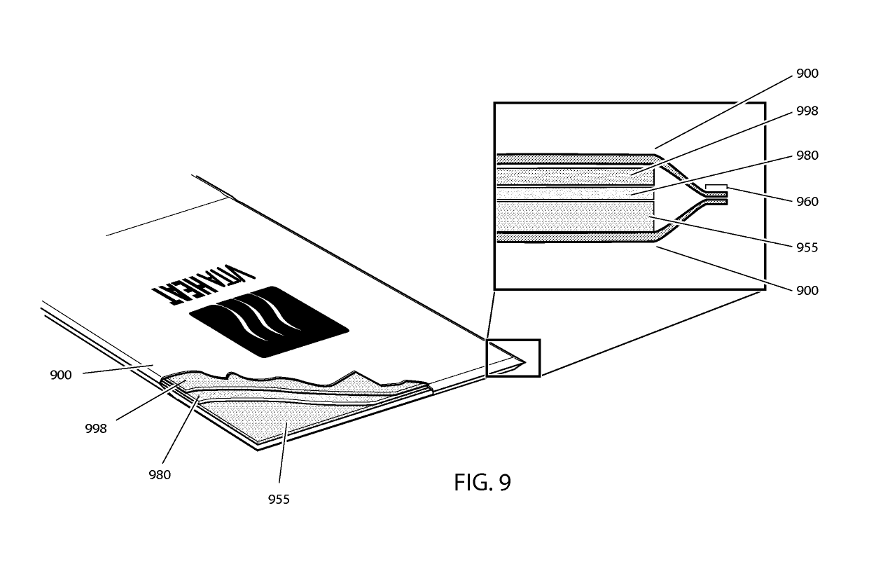

In some embodiments, as shown in FIG. 9, a heat dissipating layer (998) may be disposed on the heating element (980), which may be any of the heating elements described herein. The heat dissipating layer (998) may comprise, for example, conductive foam and/or gel. In some embodiments, such as when electric current is being delivered to the entire heating element, the surface of the heating pad may not be a uniform temperature due to uneven heating of the heating element. However, with a heat dissipating layer (998) disposed on the heating element, heat can be dissipated and evenly distributed across the surface of the heating pad such that the surface of the heating pad comprises consistent, uniform temperatures. In some embodiments, the heat dissipating layer (998) is disposed between the heating element (980) and the cover (900). As with other embodiments disclosed herein, the pad may additionally comprise an insulating pad (955) and a radio-frequency welded flange (960).

In some embodiments, the heat dissipating layer comprises a laminate of a nylon/spandex fabric on one side, polyurethane foam in the middle, and nylon/spandex loop fabric on the other side. An example of such a material is the AirFlex 2 from Eastex Products, Inc. This heat dissipating layer may be placed on top of the heating element with the loop fabric facing down (the heating element) and the woven fabric facing up (towards the patient). The thickness of the heat dissipating layer is not particularly limited and in some embodiments, it is about 3 mm thick.

In other embodiments, the heat dissipating layer may comprise polyurethane foams having a thickness of about 1/16 inch to about ⅛ inch. The heat dissipating layer may also comprise semi-conductive materials in some embodiments. Any semi-conductive materials may be used and illustrative, non-limiting examples include semi-conductive foams, films, and rubber sheets. If rubber sheets or the foregoing semi-conductive materials were used, not only would they be effective in dissipating heat, they could also function to electrostatic discharge shielding.

The controller, the heating pad, thermocouples, pressure sensors, temperature change sensors, and the transducers may be in communication with one another. In one embodiment, as shown in FIG. 1, an electrical cord (130) may link the heating pad and the controller to provide a wired connection. In some embodiments, each component of the heating system may comprise a transceiver. For example, the controller may comprise a transceiver (106) and the heating pad may comprise a transceiver (107). In FIG. 1, the transceiver (106) is shown as disposed on the outside of the controller housing, however, in other embodiments, the transceiver (106) may be disposed within the controller housing such that it is not visible when viewing the controller from the outside. Also, the transceiver (107) is shown as disposed on an outside surface of the heating pad, however, in some embodiments, the transceiver (107) may be disposed underneath the cover of the heating pad. The transceivers may be any type of devices configured for communication. The transceivers may communicate, for example, using wireless or radio frequency protocols.

Thus, in some embodiments, the warming system may comprise radio-frequency identification (RFID) capabilities. A RFID identification system uses readers to identify certain objects comprising tags. The readers may be two-way radio transmitter-receivers, which can send a signal to the tag and read its response. In one embodiment, as can be seen in FIG. 1, the controller may comprise a RFID reader (108) and the heating pad may comprise a RFID tag (109). Although the RFID reader (108) is shown as disposed on the outside of the controller housing in FIG. 1, in other embodiments, the RFID reader (108) may be disposed within the controller housing such that it is not visible when viewing the controller from the outside. Also, the RFID tag (109) is shown as disposed on an outside surface of the heating pad, however, in some embodiments, the RFID tag (109) may be disposed underneath the cover of the heating pad.

In some embodiments, the RFID reader and the RFID tag must be compatible in order for them to communicate. If the RFID tag on the heating pad is not compatible with the RFID reader in the controller, the controller will not be able to control the heating pad, such as increasing or decreasing the electric current being supplied thereto. Moreover, in some embodiments, the RFID tag may be time dated. In these embodiments, the tag may be dated for a certain time period, for example, two years, and after two years, the tag will no longer be compatible with the RFID reader. Such a configuration will help ensure that heating pads, or any other devices comprising the RFID tags, will only be used for their intended lifetime.

In some embodiments, the controller and/or heating pad may comprise a RFID reader and a RFID tag may be placed in any other device that may be used near the heating pad. For example, a patient may be lying on the heating pad and there may be a surgical tool or a sponge, for example, in the area. The surgical tool and/or sponge may comprise a RFID tag and the controller and/or heating pad may comprise a reader to provide existence and/or location data for the tool/sponge.

In some embodiments, the transceivers may comprise Bluetooth™ capabilities, such that the components of the system, for example, the transducers, controller and other heating pad components, can be paired and communicate with each other. The communication is accomplished wirelessly through short-range networks called piconets. Other devices comprising Bluetooth™ capabilities, such as additional controllers, may also communicate in the piconet network with the heating pad such that more than one controller may be used to control the heating pad.

In one embodiment, a controller comprising Bluetooth™ capabilities may be used to view a temperature reading of the heating zones, whereby a temperature of a plurality of different zones on the heating pad may be seen on an interface of the controller.

As used herein, the term “controller” refers to an electronic device having components such as a processor, memory device, digital storage medium, a communication interface including communication circuitry operable to support communications across any number of communication protocols and/or networks, a user interface (e.g., a graphical user interface that may include cathode ray tube, liquid crystal display, plasma display, touch screen, or other monitor), and/or other components. The controller is preferably operable for integration with one or more application-specific integrated circuits, programs, computer-executable instructions or algorithms, one or more hard-wired devices, wireless devices, and/or one or more mechanical devices. Some or all of the controller system functions may be at a central location, such as a network server, for communication over a local area network, wide area network, wireless network, internet connection, microwave link, infrared link, wired network (e.g., Ethernet) and the like. In addition, other components such as a signal conditioner or system monitor may be included to facilitate signal transmission and signal-processing algorithms.

In certain aspects, the controller comprises appropriate software to receive data from one or more thermocouples or temperature sensors, for example, to determine if the temperature in one or more zones of the heating pad is within an acceptable range. The software can also allow the controller to determine appropriate actions that should be taken to remedy an undesired temperature and/or undesired temperature rate of change, such as increasing or decreasing current to the heating element or a specific portion of the heating element.

Data transmission in the present application is accomplished using any suitable device, and across any number of wired and/or wireless networks, including, as illustrative examples, WiFi, WiMAX, Ethernet, cable, digital subscriber line, Bluetooth™, Zigbee™, cellular technologies (e.g., 2G, 3G, Universal Mobile Telecommunications System (UMTS), GSM, Long Term Evolution (LTE), or more)etc. Any suitable interface standard(s), such as an Ethernet interface, wireless interface (e.g., IEEE 802.11a/b/g/x, 802.16, Bluetooth, optical, infrared, radiofrequency, etc.), universal serial bus, telephone network, the like, and combinations of such interfaces/connections may be used. As used herein, the term “network” encompasses all of these data transmission methods. Any of the described devices may be connected to one another using the above-described or other suitable interface or connection.

The presently disclosed controller may be used to adjust the electric current being applied to the heating element or a portion thereof. It may also be used to turn the system on and off. In some embodiments, the controller adjusts the current based on input provided to the controller from a user. For example, a user can depress a button on the controller to increase the current or a button on the controller to decrease the current. In other embodiments, the controller may automatically adjust the amount of current being supplied to the heating element based on information received from the thermocouples, temperature change sensors, and/or pressure sensors disposed on the heating pad, heating element, and/or substrate. All such components may be in electronic communication with the controller and the communication may be wired or wireless. This creates a closed loop system in which the controller is much better adapted to provide consistent, controllable, and precise warmth through the heating pad.

The controller may also provide a power feed to the heating element to create a uniform heat pattern across the substrate/heating pad or the controller may only supply power to certain portions of the heating element, such as those portions in close proximity to a patient or body part of a patient.

In some embodiments, the controller comprises a variety of different safety features. For example, the controller may comprise dual shutdown mechanisms (one in hardware and another in software) to cut power in the event that excessive electric current flows to the heating element. Likewise, there may be dual mechanisms to cut power if any of the thermocouple or temperature sensor readings exceed a preset threshold, such as about 41° C. In some aspects, if the electric cord connecting the heating pad and controller is disconnected, the software can detect the disconnection and cease operation of the warming system. The controller may also comprise logic to detect extreme temperature differences between zones, such as about 3° C. or more, to detect an unusually low temperature, such as less than about 68° F., to detect an unusually high temperature, such as higher than about 41° C. in one or more zones, and/or to detect extreme temperature changes, such as about 2° C. per minute or more, across the pad. In some embodiments, if one of these events was detected by the controller, the controller may automatically shut down the system.

In some embodiments, as can be seen in FIG. 1, the controller (105) may be disposed in a housing that optionally includes an integrated handle (135) to hang from a bedrail or similar location. The user interface (140) of the controller can be specifically configured to suit the needs of the intended operator. In some embodiments, the operator is presented with a very simple set of controls. For example, the controller may comprise an “on/off” button and it may also comprise two additional buttons for increasing or decreasing the temperature setting of the heating pad. In some embodiments, the controller may comprise an LED bar, such as a 10-segment LED bar, between or next to the two temperature buttons to show the relative heat setting. The higher the heat setting, for example, the more segments of the LED bar are illuminated. In such an embodiment, since there are no characters or digits on the user interface, a person with limited eyesight can ascertain the current setting and locate the buttons to adjust the setting, in both dark and lighted rooms, since each button may be illuminated.

In some embodiments, the controller may be operatively coupled to the heating element and to the power source. In such embodiments, a continuous electrical path is formed between the power source and the heating element. Moreover, a continuous signal path may be formed between a thermocouple and the controller.

In certain embodiments, the controller need not be plugged into an electrical outlet. Instead, the controller may comprise a battery, which powers the warming system. In further embodiments, the controller comprises a battery and may be plugged into an electrical outlet. In such embodiments, the electrical outlet may be used to charge the battery.

While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a device” is intended to include “at least one device” or “one or more devices.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges (including all fractional and whole values) subsumed therein.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A warming system comprising:
a heating pad and a controller, wherein the heating pad comprises:
a substrate comprising a heating element disposed under a covering, wherein the heating element comprises conductive ink and the substrate comprises one or more thermocouples, wherein the one or more thermocouples have diameters less than about 0.13 mm; and
an electric cord in communication with the heating element and the controller, the controller comprising a battery and being configured to increase or decrease an amount of an electric current being supplied to the heating element;
wherein the substrate comprises one or more discrete lines of the conductive ink forming a pattern and each line of conductive ink has a thickness of less than about 12 μm.

2. The warming system of claim 1, wherein the conductive ink comprises silver ink and carbon ink fixedly disposed on the substrate.

3. The warming system of claim 1, further comprising a heat dissipating layer.

4. The warming system of claim 1, wherein the system is portable.

5. The warming system of claim 1, wherein the substrate comprises one or more pressure sensors and/or one or more temperature change sensors connected via a conduit to one or more transducers, the transducers being in electronic communication with the controller, wherein the one or more pressure sensors comprise air pouches.

6. The warming system of claim 1, wherein the heating pad is radio translucent.

7. The warming system of claim 1, wherein each of the one or more thermocouples is independently in communication with the controller.

8. The warming system of claim 1, wherein the controller and the heating pad comprise transceivers.

9. The warming system of claim 1, wherein the controller comprises a radio-frequency identification reader and the heating pad comprises a radio-frequency identification tag.

10. The warming system of claim 1, wherein the heating pad comprises one or more openings therethrough.

11. The warming system of claim 1, wherein the pattern of conductive ink comprises lines, squares and/or rectangles.

12. The warming system of claim 1, wherein the controller comprises a power cord that may be plugged into an electrical outlet.

13. A method of warming a body part of a patient comprising:
providing a warming system, wherein the warming system comprises a heating pad and a controller, the heating pad comprising a substrate comprising a heating element disposed under a covering, the heating element comprising one or more thermocouples, the warming system further comprising an electric cord in communication with the heating element and the controller, the controller comprising a battery and being configured to increase or decrease an amount of an electric current being supplied to the heating element, the substrate comprising one or more pressure sensors connected via a conduit to one or more transducers, the transducers being in electronic communication with the controller;
contacting a portion of the heating pad with the body part of the patient, the body part of the patient applying pressure to one or more of the pressure sensors causing a pressure change wherein air from within the one or more pressure sensors flows through the conduit and into the one or more transducers;
sending a communication from the one or more transducers to the controller to alert the controller of the pressure change; and
supplying electric current only to a portion of the heating element where the pressure change was detected and not to any other portion of the heating element.

14. The method of claim 13, wherein the method is carried out in a hospital, surgical center, or an emergency medical system vehicle.

15. The method of claim 13, wherein the controller comprises a power cord, the power cord being plugged into an electrical outlet, thereby charging the battery of the controller while the method is carried out.

16. The method of claim 13, wherein the heating element comprises a conductive ink, a flexible polymeric sheet doped with one or more metals, or a fabric impregnated with one or more metals, the one or more metals and conductive ink comprising silver and carbon.

17. A warming system comprising:
a heating pad and a controller, wherein the heating pad comprises:
a substrate comprising a heating element disposed under a covering, wherein the heating element comprises one or more thermocouples; and
an electric cord in communication with the heating element and the controller, the controller comprising a battery and being configured to increase or decrease an amount of an electric current being supplied to the heating element;
wherein the substrate comprises one or more pressure sensors connected via a conduit to one or more transducers, the transducers being in electronic communication with the controller.

18. The warming system of claim 17, wherein the heating element comprises a conductive ink, a flexible polymeric sheet doped with one or more metals, or a fabric impregnated with one or more metals, the conductive ink and one or more metals comprising silver and carbon.

19. The warming system of claim 17, wherein the conductive ink is fixedly disposed on the substrate and comprises grooves to allow for flexibility.

20. The warming system of claim 17, wherein the controller comprises a radio-frequency identification reader and the heating pad comprises a radio-frequency identification tag.

21. The warming system of claim 17, wherein the controller is configured to stop the electric current from being transported to the heating element if a temperature of the heating pad drops below about 68° F., rises above about 41° C., and/or changes by about 2° C. per minute or more across the heating pad.
